SEQUENCE LISTING (34 sequences)

SEQ ID NO: 1 - protein, 8 aa, Chlamydia suis
AESAPEEP

SEQ ID NO: 2 - protein, 8 aa, Chlamydia suis
SSSTTTAS

SEQ ID NO: 3 - protein, 8 aa, Chlamydia suis
ASQSTSSD

SEQ ID NO: 4 - protein, 8 aa, Chlamydia suis
TRSASAES

SEQ ID NO: 5 - protein, 8 aa, Chlamydia suis
APENNRQG

SEQ ID NO: 6 - protein, 8 aa, Chlamydia suis
QASSTENQ

SEQ ID NO: 7 - protein, 8 aa, Chlamydia suis
NDNQSASE

SEQ ID NO: 8 - protein, 8 aa, Chlamydia suis
ASENDQSS

SEQ ID NO: 9 - protein, 8 aa, Chlamydia suis
SGQEGGNE

SEQ ID NO: 10 - protein, 8 aa, Chlamydia suis
EGSASNEP

SEQ ID NO: 11 - protein, 8 aa, Chlamydia suis
SGSSGSGQ

SEQ ID NO: 12 - protein, 8 aa, Chlamydia suis
GDNNAGSG

SEQ ID NO: 13 - protein, 8 aa, Chlamydia suis
SSSSSAPA

SEQ ID NO: 14 - protein, 10 aa, Chlamydia suis
QEENEETDED

SEQ ID NO: 15 - protein, 28 aa, Chlamydia suis
AESAPEEPSSSTTTASQSTSSDQPQNNA

SEQ ID NO: 16 - protein, 53 aa, Chlamydia suis
VDKSETQNPSGGSGTGDSSDSSEAEGSSGSSNDSANNSSGGDSNGVSAAAQAA

SEQ ID NO: 17 - protein, 36 aa, Chlamydia suis
GSQGTEQDSQEGSPGSTGSQESATNSASSQQSSIAS

SEQ ID NO: 18 - protein, 9 aa, Chlamydia suis
APENNRQGD

SEQ ID NO: 19 - protein, 34 aa, Chlamydia suis
QASSTENQNDNQSASENDQSSGGGSSSPTSPQSP

SEQ ID NO: 20 - protein, 16 aa, Chlamydia suis
SGQEGGNEEGSASNEP

SEQ ID NO: 21 - protein, 16 aa, Chlamydia suis
SGSSGSGQGDNNAGSG

SEQ ID NO: 22 - protein, 9 aa, Chlamydia suis
SSSSSAPAS

SEQ ID NO: 23 - protein, 22 aa, Chlamydia suis
AESAPEEPSSSTTTASQSTSSD

SEQ ID NO: 24 - protein, 21 aa, Chlamydia suis
QASSTENQNDNQSASENDQSS

SEQ ID NO: 25 - protein, 18 aa, Chlamydia suis
QEENEETDEDSSLDSRNT

SEQ ID NO: 26 - protein, 41 aa, Chlamydia suis
AESAPEEPSSSTTTASQSTSSDQPQNNAIALRSFLYSLQTE

SEQ ID NO: 27 - protein, 86 aa, Chlamydia suis
LTFSKNAVKVTFVDKSETQNPSGGSGTGDSSDSSEAEGSSGSSNDSANNSSGGDSNGVSAAAQAAAFSRFLSASTSTDPQPGEAEN

SEQ ID NO: 28 - protein, 76 aa, Chlamydia suis
GSQGTEQDSQEGSPGSTGSQESATNSASSQQSSIASARLTQLSLTRSASAESGGSQSQENTSQQNVQASNDTQQGQ

SEQ ID NO: 29 - protein, 69 aa, Chlamydia suis
APENNRQGDASENHQQGEQASSTENQNDNQSASENDQSSGGGSSSPTSPQSPTTEVIKPVVGRGGAVYT

SEQ ID NO: 30 - protein, 53 aa, Chlamydia suis
SGSEEASEASSGQEGGNEEGSASNEPQASPVRSSSEDRAQEAGSDSTPSSETN

SEQ ID NO: 31 - protein, 25 aa, Chlamydia suis
AIPADNNSVTATVSDSGTPSTTPDP

SEQ ID NO: 32 - protein, 40 aa, Chlamydia suis
PEGSGGSAASNTGSGSSGSGQGDNNAGSGSSSSTSTDNSN

SEQ ID NO: 33 - protein, 16 aa, Chlamydia suis
SSSSSAPASSSTVPAA

SEQ ID NO: 34 - protein, 8 aa, Chlamydia suis
SQQSSIAS

(19) United States
(12) Patent Application Publication
(10) Pub. No.: US 2020/0240989 A1
(43) Pub. Date: Jul. 30, 2020

(54) CHLAMYDIA SUIS DIAGNOSIS

(71) Applicant: UNIVERSITEIT GENT, Gent (BE)

(72) Inventor: Daisy VANROMPAY, Oosterzele (BE)

(21) Appl. No.: 16/754,385

(22) PCT Filed: Oct. 11, 2018
(86) PCT No.: PCT/EP2018/077680

§ 371 Date: Apr. 8, 2020

(30) Foreign Application Priority Data
Oct. 13, 2017 (EP) 17196280.6

(51) Int. Cl. G01N 33/569; C07K 7/06; C07K 7/08; C07K 14/295

(52) U.S. Cl. CPC G01N 33/56927; C07K 7/06; G01N 2800/26; C07K 14/295; G01N 2469/20; C07K 7/08

(57) ABSTRACT

The present invention relates to a method for detecting and diagnosing Chlamydia suis infections in a subject, and a diagnostic kit therefor.

### FIELD OF THE INVENTION

The present invention relates to a method for detecting and diagnosing Chlamydia suis infections in a subject, and a diagnostic kit therefor.

### BACKGROUND OF THE INVENTION

Chlamydia suis (C. suis) is an obligate intracellular Gram-negative bacterium, belonging to the order of Chlamydiales. The pig is the only known natural host of C. suis. Chlamydia suis is currently considered to be the most prevalent chlamydial species in pigs but pigs also can become infected by C. pecorum, C. abortus and C. psittaci (reviewed by Schautteet and Vanrompay, 2011). Chlamydia suis in pigs has been associated with asymptomatic infections but also with a variety of clinical symptoms such as conjunctivitis, rhinitis, pneumonia, enteritis, reproductive disorders such as irregular return to oestrus, early embryonic dead in inseminated sows and inferior semen quality in boars (decrease of sperm cell motility and death of sperm cells) (reviewed by Schautteet et al., 2011; Schautteet et al., 2013; Chahota et al., 2017). Differences in clinical symptoms and pathology caused by C. suis are thought to be due to a high degree of genetic diversity in C. suis. The latter has recently been proven by Joseph et al., (2016), who performed the first comparative genomic analysis of C. suis, by whole genome alignment of 12 C. suis strains. Joseph et al., (2016) and more recently also by others, found evidence of DNA exchange by homologous recombination in C. suis. In fact, C. suis had the highest recombination rate of all Chlamydia species studied to date. Moreover, Joseph et al., (2016) also found evidence of genetic exchange between C. suis and other Chlamydia species.

Chlamydia suis infections could be successfully treated with tetracyclines until the appearance of a tetracycline resistant (Tc^{R}) phenotype, which was first isolated on pig farms in Iowa and Nebraska (Andersen and Rogers, 1998). Soon thereafter, tetracycline resistant C. suis strains appeared in other countries including Belgium, Cyprus, Germany, Israel, Italy, Switzerland and The Netherlands (Di Francesco et al., 2011; Borel et al., 2012; Schautteet et al., 2013; Wanninger et al., 2016).

The emergence of Tc^{R} C. suis strains raises considerable concern because C. suis shares 79.8% average nucleotide identity with the human pathogen C. trachomatis. C. trachomatis is the leading cause of sexually transmitted diseases (STD) and preventable blindness (trachoma) worldwide. Recently, Dean et al., (2013), found C. suis mRNA in the eyes of Nepalese trachoma patients. Chlamydia suis DNA and viable organisms were also recovered from the eyes of Belgian slaughterhouse employees (De Puysseleyr et al. 2014a) and from the eye, nose, throat and stool of Belgian pig farmers (De Puysseleyr et al. 2017). As far as we know, Tc^{R} C. suis phenotypes have not yet been found in humans.

Nowadays, C. suis infections are often unnoticed because diagnosis is still not routinely performed in veterinary diagnostic laboratories as: i) tetracycline resistant C. suis strains relatively recently emerged, ii) C. suis strains are hard to culture and iii) C suis-specific molecular diagnostic techniques, such as real-time PCR and DNA micro-array, only became available in recent years (Sachse et al., 2005; De Puysseleyr et al., 2014b; Lis et al., 2014) and are often regarded as too expensive by the pork industry. Moreover, C. suis is sometimes found in association with other pathogens (the usual suspects), which are mostly more easily to detect.

Serological tests to detect the presence of anti-C. suis antibodies are able to actually prove the presence of an immunological response and thus an infection. Unfortunately, at present, no assay for C. suis specific serodiagnosis is available.

Antibody detection assays which came first available for veterinary use detected antibodies against whole chlamydia organisms or against the surface exposed chlamydia lipopolysaccharide (LPS) (Sachse et al., 2009). Lack of Chlamydia specificity, resulted in the development of tests detecting antibodies against the recombinant Chlamydia full-length major outer membrane protein (MOMP) (Hoelzle et al., 2004; Verminnen et al., 2006). Later on, other surface exposed antigens like outer membrane protein 2 (Omp2), polymorphic membrane proteins (Pmp's) and virulence associated antigens like for instance translocated actin-recruiting phosphoprotein (Tarp), inclusion membrane proteins (Inc's), Chlamydia protease-like activity factor (CPAF) and secreted inner nuclear membrane-associated Chlamydia protein (SINC) were explored for use in serology, with varying degrees of success in terms of sensitivity and specificity (Longbottom et al., 2002; Forsbach-Birk et al., 2013; Rahman et al., 2015). Development of serological tests for Chlamydiae of veterinary interest now focuses towards the use of species-specific peptides of surface exposed antigens or of virulence associated antigens as it proved to be successful for C. trachomatis diagnosis in humans, leading to several commercial available ELISA's based on the use of highly immunogenic C. trachomatis species-specific MOMP peptides (the C. trachomatis-IgG-pELISA plus Medac assay, Medac, Wedel, Germany; the SeroCT-IgG ELISA, Savyon Diagnostics, Ashdod, Israel; the C. trachomatis IgG EIA Ani Labsystems, Vantaa, Finland). In 2015, a test based on a recombinant fragment of polymorphic membrane protein 12G (Pmp12G) in an ELISA format became commercially available for detecting antibodies against C. abortus in sheep (Longbottom et al., 2002; Forsbach-Birk et al., 2013). However, in some cases, cross-reactivity between Chlamydia species can hinder interpretation of results (Donati et al. 2009). Hoelzle et al. demonstrated the suitability of the full length recombinant MOMP proteins of C. suis, C. abortus and C. pecorum in ELISA assays to identify the infecting chlamydial species (Hoelzle et al. 2004). However, until now, this approach has not been further verified in animals in the field. Furthermore, a recombinant protein fragment of the Pmp90 was successfully used for serological diagnosis of C. abortus infections (Longbottom et al. 2001). The Pmps are considered as important virulence factors and several studies demonstrated the induction of an immune response (Grimwood and Stephens 1999; Niessner et al. 2003; Wehrl et al. 2004) and even protective immunity (Cevenini et al. 1991). Tan et al. (2009) demonstrated that the Pmps elicit various serologic responses in C. trachomatis-infected patients. However, differences in the strengths and specificities of the Pmp subtype-specific antibody reactivity related to gender and clinical outcome were observed. This indicates that the Pmp gene family forms the basis of a mechanism of antigenic variation. The PmpD protein of C. abortus was recognized as a major antigen using sera of experimentally infected ewes. In addition, also the MOMP protein and the translocated actin recruitment protein (Tarp) were identified as reactive proteins in this study (Marques et al. 2010; Forsbach-Birk et al. 2013). The Tarp protein is also predominantly recognized by antibodies from humans infected with C. trachomatis (Wang et al. 2009). WO2016/150930 discloses the identification of two antigenic determinant peptides derived from the MOMP and PmpC of C. suis useful in serodiagnosis.

Specific detection of anti-C. suis antibodies can provide the evidence for an existent infection in pigs and humans, an essential factor in the study of the zoonotic transfer of this microbe. To date, no sensitive C. suis specific commercial assay is available for animal or human serodiagnosis. Seroprevalence studies in pigs are based on detection of antibodies against LPS, MOMP and whole EB preparations and serological cross-reactions with antibodies against other chlamydial species or other pathogens do occur (Schautteet and Vanrompay 2011).

There is thus currently still a need for antigenic peptides that are useful in the detection or diagnosis of Chlamydia suis in a subject.

### SUMMARY OF THE INVENTION

The present invention encompasses a C. suis specific and antigenic peptide, in particular for serodiagnostic use, a kit comprising and a method using said peptide.

Thus, in one embodiment the invention relates to a method and kit for detecting and/or diagnosing Chlamydia suis infections in a subject using at least one peptide comprising an amino acid sequence selected from the group consisting of SEQ ID NO: 1-13 as antigenic determinant.

A further embodiment of the present invention relates to isolated peptides comprising or consisting essentially of one or more of the amino acid sequences as represented by SEQ ID NO: 1-13, or a variant thereof. In a specific embodiment, the peptide comprising an epitope is about and between 7 to 30 amino acids long.

In a further embodiment, the invention relates to a method for diagnosing a Chlamydia suis infection in a subject. In particular, the invention comprises a method for detecting the presence of Chlamydia suis in a subject, comprising
providing a biological sample from a subject, and
analyzing the sample for the presence of antibodies against one or more of the epitopes characterized by an amino acid sequence given in SEQ ID NO 1 to 13, or a sequence substantially identical thereto, or against peptides comprising said epitopes,
whereby the presence of antibodies against one or more of the epitopes or peptides comprising them is indicative for the presence of Chlamydia suis in the subject.

In a further embodiment, the invention provides a combination, e.g. as a fusion polypeptide or as separate peptides, and a method or use thereof as described herein, of at least two, three, four, five or more epitopes or peptides as described herein, e.g any combination of sequences selected from the group represented by SEQ ID NO: 1-34, in particular SEQ ID NO: 1-24, more in particular SEQ ID NO: 1-13. In a particular embodiment, the peptide, kit or method as described herein do not comprise (use of) the full length PmpC protein of Chlamydia suis.

In some embodiments of this aspect of the invention the method comprises one or all of the following steps:
a surface is provided, where the epitope(s)/peptide(s) as disclosed herein are attached;
blood or other (fluid) sample obtained from a subject is contacted, optionally after removal of irrelevant components, with the surface under conditions allowing for the specific binding of an antibody to an epitope;
detecting the antibody-peptide complex and/or determining the amount of the bound antibody;
optionally, a washing step is inserted between the first and the second step to remove any sample components that did not interact with the surface. Another washing step may follow the second contacting step and precede the determination.

Preferably, the biological sample is from a mammal, in particular a human or a pig.

In a particular embodiment, the detection of antibodies is conducted using an immunoassay, such as an ELISA. Typically, the biological sample to be tested is a smear or body fluid, preferably mucosal secretions or blood, in particular serum.

Diagnosis is possible at an early stage of the disease, during therapy, as well as after clearance of the infection. In addition, the peptides can be useful to differentiate a vaccinated from an infected subject (DIVA principle). Another embodiment of the present invention relates to Chlamydia suis antigenic peptides and a kit comprising these peptides, in particular for the use in a method according to the present invention, namely for detecting the presence of C. suis in a subject. Preferably, the test kit is an ELISA.

The invention also relates to the use of the peptide(s) or kit in the diagnosis of C. suis infection in a subject, and to a method of preparing said peptide(s) or kit.

### DESCRIPTION OF THE INVENTION

As used herein, the singular forms “a”, “an”, and “the” include both singular and plural referents unless the context clearly dictates otherwise. The terms “comprising”, “comprises” and “comprised of” as used herein are synonymous with “including”, “includes” or “containing”, “contains”, and are inclusive or open-ended and do not exclude additional, non-recited members, elements or method steps. The term “about” as used herein when referring to a measurable value such as a parameter, an amount, a temporal duration, and the like, is meant to encompass variations of +/−20% or less, preferably +1-10% or less, more preferably +/−5% or less, of and from the specified value, insofar such variations are appropriate to perform in the disclosed invention. It is to be understood that the value to which the modifier “about” refers is itself also specifically, and preferably, disclosed. Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any >3, >4, >5, >6 or >7 etc. of said members, and up to all said members. All references, and teachings specifically referred to, cited in the present specification are hereby incorporated by reference in their entirety. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. Reference throughout this specification to “one embodiment” or “an embodiment” means that a particular feature, structure or characteristic described in connection with the embodiment is included in at least one embodiment of the present invention.

The present invention relates to peptides, a kit and a method for diagnosing or detecting Chlamydia suis infection in a subject. The method is based on the detection of antibodies specifically recognizing immunoreactive peptides derived from the polymorphic membrane protein C (PmpC) of Chlamydia suis. As used herein, the PmpC protein is meant to include any homolog, isolated or artificial sequence that is substantially identical to the corresponding or encoded PmpC protein as found in the C. suis MD56 reference genomic sequence with Genbank accession no. AYKJ00000000.

The detection of antibodies in animal chlamydial infections has multiple purposes, i.e. confirmation of clinical disease or confirmation of the presence or absence of infection, performance of epidemiological surveys to estimate the prevalence of infection, or the determination of immune status after vaccination.

Since C. suis and C. trachomatis are phylogenetically closely related and the availability of C. suis sequence information is rather limited, the selection of a specific antigen is rather complex. Therefore, the present invention focused on the identification of peptide antigens to minimize cross-reactions to other pathogens. Chlamydia Pmps are important virulence factors and candidate antigens for serodiagnosis. We identified 9 Pmps (PmpA to I) in C. suis strain MD56 using a recently developed Hidden-Markov model. PmpC was a promising candidate for the development of a C. suis-specific antibody assay as the protein was absent in C. abortus, C. pecorum and C. psittaci which also infect pigs and as the protein contained C. suis-specific amino acid regions, absent in C. trachomatis PmpC. Previously, an immunodominant B cell epitope in C. suis PmpC was identified using experimental sera and field sera from pigs being C. suis PCR positive in the rectum (WO2016/150930). Unexpectedly, additional epitopes in said PmpC were identified in the present invention by using field sera collected in the slaughterhouse from healthy pigs being C. suis positive in the vagina and by using field sera of clinically affected pigs being C. suis PCR and/or C. suis culture positive in either their reproductive tract or their conjunctiva. The additional epitopes revealed higher OD values for these sera than the previously identified B cell epitope. Thus, the formerly identified epitope (SQQSSIAS; SEQ ID NO:34) can be used for the identification of C. suis carriers (C. suis thought to be resident in the gut) and for confirming C. suis as the etiological agent in diarrhea. The epitopes of the present invention (one or more of SEQ ID 1 to 13) can be used for confirming C. suis as the etiological agent in reproductive failure and conjunctivitis.

The usefulness of one or more of the epitopes represented by SEQ ID 1 to 13 for the purpose mentioned, was confirmed by a pin-peptide ELISA (Pepscan System, Lelystad, The Netherlands) and by an alternative serological assay, namely a peptide micro array (JPT Innovative Peptide Solutions; Berlin Germany) using the sera described in Table 1 and 2.

The present invention thus relates to epitopes derived from the PmpC of C. suis which are specifically recognized by antibodies. In particular, said epitopes consist of an amino acid sequence identical or substantially identical to the following sequences:

| PmpC region | Amino acid sequence | SEQ ID NO |
|---|---|---|
| 126-134 | AESAPEEP | 1 |
| 135-143 | SSSTTTAS | 2 |
| 142-150 | ASQSTSSD | 3 |
| 498-506 | TRSASAES | 4 |
| 549-557 | APENNRQG | 5 |
| 568-576 | QASSTENQ | 6 |
| 577-585 | NDNQSASE | 7 |
| 583-591 | ASENDQSS | 8 |
| 975-983 | SGQEGGNE | 9 |
| 984-992 | EGSASNEP | 10 |
| 1643-1651 | SGSSGSGQ | 11 |
| 1652-1660 | GDNNAGSG | 12 |
| 1781-1789 | SSSSSAPA | 13 |

In a particular embodiment, the epitope of the invention is not SQQSSIAS (SEQ ID NO:34).

In a further embodiment, the invention relates to a peptide, in particular an immune reactive peptide, comprising one, two, three, four or more of the epitopes of the present invention. As used herein “immune reactive peptides” or “peptide antigens” refers to (poly)peptides of at least about 7 amino acid residues, like 6, 7, or 8, and up to 35, 40, 45, 50 or 60 amino acid residues long. Said peptides are typically about 7 to 30 amino acids long, in particular about 8 to 25 amino acids, more in particular about 8 to 20, and even more particular about 8 to 15 amino acids long, including all integers in between. The immune reactive peptides as disclosed herein may be used singly or in combination. In a preferred embodiment, the current invention encompasses a peptide consisting essentially of or consisting of the amino acid sequence disclosed in the group selected from SEQ ID NOs: 1-24, in particular SEQ ID NO: 1-13, and the use thereof. The peptides of the invention may be used alone or, preferably, in combination, e.g. in a kit or assay, either coupled to each other or individually.

Exemplary peptides are:

(SEQ ID NO: 14)
QEENEETDED,
(SEQ ID NO: 15)
AESAPEEPSSSTTTASQSTSSDQPQNNA,
(SEQ ID NO: 16)
VDKSETQNPSGGSGTGDSSDSSEAEGSSGSSNDSANNSSGGDSNGVSAAA
QAA,
(SEQ ID NO: 17)
GSQGTEQDSQEGSPGSTGSQESATNSASSQQSSIAS,
(SEQ ID NO: 18)
APENNRQGD,
(SEQ ID NO: 19)
QASSTENQNDNQSASENDQSSGGGSSSPTSPQSP,
(SEQ ID NO: 20)
SGQEGGNEEGSASNEP,
(SEQ ID NO: 21)
SGSSGSGQGDNNAGSG,
(SEQ ID NO: 22)
SSSSSAPAS,
(SEQ ID NO: 23)
AESAPEEPSSSTTTASQSTSSD,
and
(SEQ ID NO: 24)
QASSTENQNDNQSASENDQSS.

It will be recognized by the skilled person that, within the amino acid sequence defined herein, substitution and/or deletion of one or possibly more of the amino acids can be made without excessive decrease in the reactivity and/or specificity. Such variants, which are functionally (or substantially) equivalent to the present peptides or epitopes but contain certain amino acid residues which may be non-naturally occurring, modified and/or synthetic, are within the scope of the present invention, if they are recognized by antibodies specific to C. suis. The skilled person would be aware that the antibody binding ability of epitope analogues containing, for example, single amino acid substitutions, may be determined using a suitable scanning technique. In one embodiment, the peptides may be modified for the purposes of ease of conjugation to a carrier. For example, it may be desirable for some chemical conjugation methods to include a terminal cysteine.

A “substantially identical” sequence (optionally referred to as “variant”) is an amino acid sequence that differs from a reference sequence only by one or more conservative substitutions, as discussed herein, or by one or more non-conservative substitutions, deletions, or insertions located at positions of the sequence that do not destroy the biological function of the amino acid molecule. Such a sequence can be any integer from 75% to 99%, or more generally at least 75%, 80%, 85%, 86%, 87%, 88%, 89%, 90%, or 95%, or as much as 96%, 97%, 98%, or 99% identical at the (global) amino acid or nucleotide level to the sequence used for comparison using, for example, FASTA. For polypeptides, the length of comparison sequences may be at least 5, 10, or 15 amino acids, and up to 20, 25, 30, 40, 50 or 60 amino acids (including all integers in between). Sequence identity can be readily measured using publicly available sequence analysis software (e.g. BLAST software available from the National Library of Medicine).

Alternatively, variants may be identified by modifying one of the above peptide sequences and evaluating the antigenic properties, secondary structure and/or hydropathic nature of the modified peptide using, for example, the representative procedures described herein. A “conservative substitution” is one in which an amino acid is substituted for another amino acid that has similar properties, such that one skilled person in the art would expect the nature of the peptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. In a specific embodiment, a substantially identical sequence is derived from a Chlamydia suis strain or isolate.

The present invention also includes a “fusion polypeptide” which may comprise a linear multimer of 2, 3, 4, 5, 6, 7, 8, 9, 10 or more repeats of a single peptide or epitope, or of a combination of the above peptide or epitope monomers linked end to end, directly or with a linker sequence present between the monomer repeats.

Determining the presence of antibodies against one or more of the PmpC derived peptides or epitopes of the present invention is indicative of C. suis infection. More specific, the presence of said antibodies in a subject can be indicative for a past (whereby the infection is cleared), an early or late infection. In addition, detection of antibodies against the peptide(s) of the present invention can be useful in the serological differentiation between vaccine-induced antibodies and antibodies against the field strain (“DIVA” stands for Differentiating Infected from Vaccinated Animals), if one or more of the vaccine antigens specifically differ from the peptides of the present invention. Such a marker assay can detect antibodies against those proteins or peptides that are absent in the vaccine. As such, naturally infected animals can be detected in a vaccinated population.

In a specific embodiment, the present invention relates to a method for detecting a C. suis infection, said method comprising the steps of:

a) contacting a biological sample with at least one peptide/epitope of the invention, optionally coated on a solid phase, for a time and under conditions sufficient for the formation of an antibody/peptide complex, and
b) detecting said antibody/peptide complex, the presence of said complex indicating the presence of a Chlamydia suis antibody in said biological sample,
wherein the presence of an antibody is indicative of a C. suis infection or vaccination.

In one embodiment of the present invention, the method is an in vitro method. In a specific embodiment, the antibodies to be detected are of the IgA, IgM and/or IgG type. As such, qualitative and quantitative detection of antibodies in the biological sample directed against Chlamydia suis is possible and recommended to support in diagnosis and/or differentiation of past (IgM/IgG), acute, recent (IgM) and/or chronic infections (IgG).

In particular, it is demonstrated herein that determining the presence of antibodies against the peptides of the invention allows a specific diagnosis of C. suis infection, in particular C. suis infection in pigs having reproductive failure or conjunctivitis. Until today, no diagnostic test for C. suis infection is available.

The term “subject” refers to humans or other mammals, in particular pigs or swine, including sows, boars and piglets.

The term “biological sample” as used herein refers to a sample that may be extracted, untreated, treated, isolated, and concentrated from a subject. Suitably, the biological sample is selected from any part of the subject's body, but it is particularly preferred that the biological sample is a body fluid, preferably blood or mucosal secretions such as a mucosal swab or mucosal lavage. The mucosal surfaces of the body are thin and permeable barriers to the interior of the body because of their physiological activities in gas exchange (the lungs), food absorption (the gut), sensory activities (eyes, nose, mouth, and throat), and reproduction (uterus and vagina). In particular, the biological sample is serum of the subject to be diagnosed.

The term “diagnosing” as used herein refers to methods by which a skilled person can estimate and even determine whether or not a subject has suffered or is suffering from a given disease, disorder or condition. The skilled person makes the diagnosis on the basis of one or more diagnostic indicators, namely antibodies, the amount (including presence or absence) of which is indicator for the presence, severity, or absence of the condition. In addition, the peptides of the present invention are particular useful for developing a reliable assay that can differentiate infected from vaccinated subjects (DIVA assay) in case the vaccine antigens (substantially) differ from the peptides disclosed herein.

The term “detecting”, “determining” or “analyzing” as used herein refers to assessing the presence, absence, quantity, level or amount of the respective antibodies within the subject derived sample, including qualitative or quantitative concentration levels of said substances.

In some embodiments of the method as disclosed herein, detecting, determining or analyzing the presence of the antibodies in the sample can include binding of antibodies to the peptide antigen or epitope as disclosed herein, and then detecting either the binding event or the presence of the antibody isolated from the biological sample. Any known method may be used for the detection of peptide binding antibodies in a sample such as body fluids. Methods considered are, by way of non-limiting example, chromatography, mass spectrometry (and combinations thereof), enzymatic assays, electrophoresis and antibody-based assays, such as but not limited to EIA (Enzyme Immuno Assay), RIA (Radio Immuno Assay), Immunoblotting, ELISA (Enzyme Linked ImmunoSorbent Assay), CLIA (ChemiLuminescent Immuno Assay), CEDIA (Cloned Enzyme Donor Immunoassay), CMIA (Chemiluminescent Microparticle Immunoassay), MEIA (Microparticle Enzyme Immunoassay), FPIA (Fluorescence Polarization Immunoassay), GLORIA (Gold-Labeled, Optically read, Rapid Immunoassay), microarray analysis, fully-automated or robotic immunoassays and latex agglutination assays.

Contacting the biological sample with the peptide under conditions effective and for a period of time sufficient to allow the formation of immune complexes, is generally a matter of adding the composition to the sample and incubating the mixture for a period of time long enough for the antibodies to form immune complexes with the peptide presented. Said peptide antibody mixture can be detected by known means and methods. That is, detection of immune complex formation of peptide antibody can be achieved through the application of numerous approaches. These methods are generally based upon the detection of a label or marker, such as any radioactive, fluorescent, biological or enzymatic tags or labels of standard use in the art. Of course, one may find additional advantages through the use of a secondary binding ligand such as a second antibody or a biotine/avidine (streptavidine) ligand binding arrangement as it is known in the art.

As a typical example, the detection method comprises one or more of the following steps. In a first step, the biological sample is contacted and incubated with a immobilized capture (or coat) reagent, i.e. the peptide(s) of the invention. Immobilization conventionally is accomplished by insolubilizing the capture reagent either before the assay procedure, as by adsorption to a water-insoluble matrix or surface or non-covalent or covalent coupling (for example, using glutaraldehyde or carbodiimide cross-linking, with or without prior activation of the support with, e.g., nitric acid and a reducing agent, or afterward, e.g., by immunoprecipitation).

The solid phase used for immobilization may be any inert support or carrier that is essentially water insoluble and useful in immunometric assays, including supports in the form of, e.g. surfaces, particles, porous matrices, etc. Examples of commonly used supports include small sheets, Sephadex, polyvinyl chloride, plastic beads, and assay plates or test tubes manufactured from polyethylene, polypropylene, polystyrene, and the like including 96- or 384-well microtiter plates, as well as particulate materials such as filter paper, agarose, cross-linked dextran, and other polysaccharides. Alternatively, reactive water-insoluble matrices such as cyanogen bromide-activated carbohydrates and the reactive substrates are suitably employed for capture reagent immobilization. In one embodiment the immobilized peptide(s) is coated on a microtiter plate, and in particular the preferred solid phase used is a multi-well microtiter plate that can be used to analyze several samples at one time, e.g. a microtest 96- or 384-well ELISA plate.

The solid phase is coated with the capture reagent as defined above, which may be linked by a non-covalent or covalent interaction or physical linkage as desired. In a specific embodiment, the peptides contain an N-terminal acetyl group and are C-terminal attached to polyethylene pins via incorporation of an extra cysteine. If covalent, the plate or other solid phase is incubated with a cross-linking agent together with the capture reagent under conditions well known in the art, e.g. such as for 1 hour at room temperature. Commonly used cross-linking agents for attaching the capture reagent to the solid phase substrate include, e.g. 1,1-bis(diazoacetyl)-2-phenylethane, glutaraldehyde, N-hydroxy-succinimide esters, homobifunctional imidoesters, and bifunctional maleimides. Derivatizing agents such as methyl-3-[(p-azidophenyl)-dithio]pro-pioimi-date yield photoactivatable intermediates capable of forming cross-links in the presence of light. In one embodiment, the invention also relates to a fusion polypeptide that includes one or more of the epitopes or peptides as provided herein and a cross-linking agent or linker.

The coated plates are then typically treated with a blocking agent that binds non-specifically to and saturates the binding sites to prevent unwanted binding of the free ligand to the excess sites on the wells of the plate. Examples of appropriate blocking agents for this purpose include, e.g., gelatin, bovine serum albumin, egg albumin, casein, and non-fat milk. The blocking treatment typically takes place under conditions of ambient temperatures for about 1-4 hours, preferably about 1 to 3 hours, or overnight. After coating and blocking, the biological sample to be analyzed, appropriately diluted, is added to the immobilized phase. The final concentration of the capture reagent will normally be determined empirically to maximize the sensitivity of the assay over the range of interest. The conditions for incubation of sample and immobilized capture reagent are selected to maximize sensitivity of the assay and to minimize dissociation. Preferably, the incubation is accomplished at fairly constant temperatures, ranging from about 0° C. to about 40° C., preferably from about 20 to 37° C. The time for incubation depends primarily on the temperature, being generally no greater than about 10 hours to avoid an insensitive assay. Preferably, the incubation time is from about 0.5 to 3 hours, and more preferably 1.5-3 hours to maximize binding.

At this stage, the pH of the incubation mixture will ordinarily be in the range of about 4-9.5, preferably in the range of about 6-9, more preferably about 7-8, and most preferably the pH of the assay (ELISA) diluent is pH 7.4. The pH of the incubation buffer is chosen to maintain a significant level of specific binding. Various buffers may be employed allowing for the specific binding of an antibody to an epitope and generally include aqueous buffer systems or aqueous solutions at physiologic pH and ionic strength. Such buffers are, by way of non-limiting example, carbonate buffer, phosphate buffered saline, sodium phosphate buffer systems, Tris/HCl buffer, glycine buffer or acetate buffer. The pH of the buffer should range between 5 and 10. Salt concentrations are defined between 0 and 250 mmol/1 using sodium chloride or an equivalent salt. Buffers may be supplemented with high salt concentrations up to 1 M to avoid unwanted interactions. The particular buffer employed is not critical to the invention, but in individual assays one buffer may be preferred over another.

In a further step, which is optional, the biological sample is separated (preferably by washing) from the immobilized capture reagent to remove uncaptured molecules. The solution used for washing is generally a buffer (“washing buffer”) with a pH determined using the considerations and buffers described above for the incubation step, with a preferable pH range of about 6-9. The washing may be done three or more times. The temperature of washing is generally from refrigerator to moderate temperatures, with a constant temperature maintained during the assay period, typically from about 0-40° C., more preferably about 4-30° C. For example, the wash buffer can be placed in ice at 4° C. in a reservoir before the washing, and a plate washer can be utilized for this step.

In a next step, the immobilized capture reagent is contacted with detectable antibodies, preferably at a temperature of about 20-40° C., more preferably about 20-37° C., with the exact temperature and time for contacting the two being dependent primarily on the detection means employed. For example, when streptavidin-peroxidase and 3,3′,5,5′-tetramethyl benzidine are used as the means for detection, e.g. in one embodiment, the contacting is carried out (e.g. about 1 hour or more) to amplify the signal to the maximum. This antibody is directly or indirectly detectable. The detectable antibody may be a polyclonal or monoclonal antibody. Also the detectable antibody can be directly detectable, and in one embodiment has a colorimetric label, and in another embodiment has a flurometric label. More preferably, the detectable antibody is biotinylated and the detection means is avidin or streptavidin-peroxidase and 3,3′,5,5′-tetramethyl benzidine. The readout of the detection means can be fluorimetric or colorimetric.

In a last step of the method, the level of antibody that is now bound to the capture reagent is measured using a detection means for the detectable antibody.

In a specific embodiment, the peptides as disclosed herein are deposited onto a carrier or solid phase and exposed to blood, serum, plasma or other antibody-containing body fluid such as mucosal secretions or smears. Consequently, so prepared compositions can be employed to identify and/or characterize an antigenic response of a subject against the specific peptides, and optionally assess the kind of response, for example identification of acute, recent primo, late, persistent or chronic infection, as well as efficacy of therapy, etc.

Direct coating via passive adsorption to the polystyrene surface of microplates in alkaline conditions is less efficient for peptide antigens compared to full-length proteins. Nevertheless, several technical solutions, like synthesis of peptide-dextran conjugates (Bocher et al. 1997), the use of a streptavidin-biotinylated peptide system (Ivanov et al. 1992), peptides bound to plastic of polylethylene pins or the use of a capture antibody (sandwich ELISA), have been developed to resolve these coating difficulties. As already mentioned, diagnostic assays contemplated herein may be based on numerous well known manners of detection, including ELISA (plate-based or solid phase; sandwich or non-sandwich), pinELISA, competitive ELISA, anti-idiotypic antibodies, direct fluorescent antibody test (DFA) etc., wherein all known colorimetric and photometric (e.g., fluorescence, luminescence, etc.) or radiometric reactions are deemed suitable for use.

In a further embodiment, the present invention provides a composition, kit or diagnostic kit comprising at least one, two, three, four, five or more of the peptides or epitopes disclosed herein. Preferably, the kit comprises instructions on how to use the kit. In preferred embodiment said test kit is an ELISA.

Typically, the kit will comprise 1, 2, 3, 4, 5, 6, 7, 8, 9, 10, 11, 12 and up to all of the epitopes or peptides comprising them as disclosed herein, optionally in combination with other peptides or proteins, such as e.g. SQQSSIAS (SEQ ID NO:34), in suitable container(s), optionally bound to a solid support, such as for example a microtiter plate, a membrane, beads, dip sticks or the like. Alternatively, the support can be provided as a separate element of the kit.

Optionally, the kit according to the present invention further includes beside the peptide(s) disclosed herein a detection agent for the antibodies which may be an antibody, antibody fragment etc. If required, the kit further comprises substrate and further means for allowing reaction with an enzyme used as label for the detecting agent, which may be an antibody. The detection agent of the kit can include a detectable label that is associated with or linked to the given detecting agent, in particular, the detecting antibody. Detectable labels that are associated with or attached to a secondary binding ligand are also contemplated. Detectable labels include dyes, illuminescent or fluorescent molecules, biotin, radiolabels or enzymes. Typical examples for suitable labels include commonly known fluorescent molecules, like rhodamine, fluorescein, green fluorescent protein or luciferase, or alkaline phosphatase and horseradish peroxidase as examples for suitable enzymes.

Optionally, the kit further comprises positive and negative controls for verifying the results obtained when using the kit. The components of the kit can be packaged either in aqueous medium or lyophilised form and, in addition, the kit may comprise one or more containers allowing to conduct the detection. In addition, the test kit comprises instructions for use of the kit.

In a further embodiment, the present invention relates to the use of the peptide(s) or kit as disclosed herein in a method for diagnosing or detection of C. suis infection. Typically, the use is in vitro. The peptides can be used alone or in combination, and the combination can be a simultaneous, separate or sequential use in a method as described herein.

The invention also relates to a method for preparing the peptide(s) as disclosed herein. The peptide of the invention can be made using standard synthetic chemistry techniques, such as by use of an automated synthesizer. In the alternative, the peptide can be made from a longer polypeptide, which polypeptide typically comprises the sequence of the peptide. The peptide may be derived from the polypeptide by for example hydrolysing the polypeptide, such as using a protease; or by physically breaking the polypeptide. The peptide can also be made in a process comprising expression of a polynucleotide. The expressed polypeptide may be further processed to produce the peptide of the invention. Thus the peptide may be made in a process comprising cultivating a cell transformed or transfected with an expression vector under conditions to provide for expression of the peptide or a polypeptide from which the peptide can be made.

The following examples are set forth below to illustrate the methods, compositions, and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

### EXAMPLES

### Materials and Methods

### 1. Chlamydia spp.

Chlamydia suis (strains S45, R19 and H7), C. abortus (strain S26/3), C. psittaci (strains 98AV2129 and 92/1293) and C. pecorum (strain 1710S) were grown in cycloheximide treated McCoy cells as described previously (Vanrompay et al., 1992). Elementary bodies for experimental infections were purified from infected cells by ultracentrifugation through a discontinuous gradient of Urografin (Urografin 76%, Schering, Machelen, Belgium). Titration of the EBs was performed by determining the tissue culture infective doses₅₀ (TCID₅₀) per ml. TCID₅₀/ml is routinely used for titrating intracellular organisms like viruses and it correlates with IFU/ml.

### 2. Animal Sera

The experimental sera (Table 1) and field sera (Table 2) were grouped as follows.

Group 1 consisted of positive control sera (n=40) that were weekly collected during 8 weeks from 5 Chlamydia free conventional bred sows (Belgian Landrace) that were experimentally (vaginally) infected at the age of 9 weeks with the C. suis reference strain S45 (De Clercq et al., 2014).
Group 2 consisted of positive control sera (n=35) that were weekly collected during 3 weeks from 5 Chlamydia free conventional bred sows (Belgian Landrace) sows that were experimentally (vaginally) infected at the age of 17 weeks with C. suis strain S45 (De Clercq et al., 2014). Sera of groups 1 (n=40) and 2 (n=35) were known to be positive in an ELISA using purified C. suis S45 EBs as antigen (De Clercq et al., 2014).
Group 3 consisted of 55 negative control sera that were weekly collected during 11 weeks (from the age of 9 to 20 weeks) from 5 Chlamydia free conventional bred sows (Belgian Landrace) that served as negative controls during an experimental infection experiment with C. suis strain S45 (De Clercq et al., 2014).
Group 4 comprised of positive control sera (n=6) that were weekly collected during 3 weeks from 2 Chlamydia negative conventional bred sows (Belgian Landrace) sows that were experimentally (combined oral, vaginal and respiratory route) infected at the age of 4 weeks with either C. suis strain H7 or C. suis strain R19. Sera tested positive in an ELISA using purified C. suis S45 EBs as antigen.
Group 5 contained sera (n=9) that were weekly collected during 3 weeks from 3 Chlamydia negative conventional bred sows (Belgian Landrace) that were experimentally infected (combined oral, vaginal and respiratory route) at the age of 4 weeks with either C. abortus strain S26/3, C. pecorum strain 170S or C. psittaci strain 98AV2129. Sera tested positive in an ELISA using purified C. suis H7 or R19 EBs, C. pecorum 170S EBs or C. psittaci 98AV2129 EBs as antigen.

Group 6 contained field sera (n=20) which were collected in a Belgian pig slaughterhouse. The sera originated from 21 different farms. Group 6A comprised sera of which the vaginal swabs tested positive in a C. suis-specific real-time PCR (De Puysseleyr et al., 2014b) while their rectal swabs tested negative. Group 6B comprised sera of which the vaginal and rectal swabs tested negative in the C. suis-specific real-time PCR. These type of sera (6A and 6B) have never been tested before for B cell epitope mapping of PmpC.

Group 7 contained sera of 10 clinically affected sows from an Israeli pig farrowing to slaughter farm dealing with reproductive failure (Schautteet et al., 2013). Three of the 10 (30%) sows had been diagnosed positive for C. suis by both culture and DNA microarray.
Group 8 contained sera of 10 clinically affected boars from an Estonian farrowing to slaughter farm dealing with reproductive failure and conjunctivitis in boars and sows (Schautteet et al., 2010). Rectal swabs of all 10 boars reacted positive in the C. suis-specific real-time PCR (De Puysseleyr et al., 2014b). Conjunctival swabs also reacted positive in the C. suis-specific real-time PCR.

All sera were heat inactivated at 56° C. for 30 minutes, subsequently pre-treated with kaolin to reduce background activity in ELISA (Novak et al., 1993) and stored at −20° C. until tested.

TABLE 1
Overview of experimental porcine sera.
Experimental sera

| Group | | | | | | | Total |
|---|---|---|---|---|---|---|---|
| (No of | Type of | Infection | Chlamydia strain | Age at | | | No of |
| pigs) | control | route | (number of pigs) | infection | | Sampling regime | samples |
| 1 (5) | Positive | Vaginal | C. suis S45 (5) | 9 | weeks | Weekly for 8 weeks | 40 |
| | | | | | | p.i.^{a} | |
| 2 (5) | Positive | Vaginal | C. suis S45 (5) | 17 | weeks | Weekly for 3 weeks | 35 |
| | | | | | | p.i. | |
| 3 (5) | Negative | NA^{b} | NA (5) | NA | | Weekly, age 9 to 20 | 55 |
| | | | | | | weeks | |
| 4 (2) | Positive | Combined | C. suis H7 (1) | 4 | weeks | Weekly for 3 weeks | 6 |
| | | oral, vaginal | C. suis R19 (1) | | | p.i. | |
| | | and | | | | | |
| | | respiratory | | | | | |
| 5 (3) | Positive for | Combined | C. abortus S26/3 (1) | 4 | weeks | Weekly for 3 weeks | 9 |
| | chlamydia | oral, vaginal | C. pecorum 170S (1) | | | p.i. | |
| | Negative | and | C. psittaci 98AV2129 (1) | | | | |
| | for C. suis | respiratory | | | | | |

^{a}p.i.: post infection;
^{b}NA: not applicable

### 3. B Cell Epitope Mapping of the Selected Pmp

B cell epitope mapping was performed for the selection of C. suis-specific, immunodominant B cell epitope(s) which could be used in an immunoassay or ELISA. First, nine C. suis-specific regions (Table 4) were identified within the selected PmpC. PmpC only occurs in C. trachomatis and in C. suis. PmpC is absent in other Chlamydia species. Thus, all C. suis-specific regions in PmpC were identified by alignment of all available C. trachomatis PmpC amino acid sequences with the corresponding PmpC amino acid sequences of C. suis strain MD56 (Table 3). Subsequently, these 9 C. suis-specific regions were used for in silico B cell epitope prediction using tools provided by the Immune Epitope Database Analysis Resource (IEDB analysis resource; Vita R, Overton J A, Greenbaum J A, Ponomarenko J, Clark JD, Cantrell J R, Wheeler D K, Gabbard J L, Hix D, Sette A, Peters B. The immune epitope database (IEDB) 3.0. Nucleic Acids Res. 2014 Oct. 9). This resource provides a collection of six tools for the prediction of linear epitopes from protein sequences [Chou and Fasman Beta-Turn prediction (Chou and Fasman, 1978), Emini surface Accessibility prediction (Emini et al., 1985), Karplus and Schulz Flexibility prediction (Karplus et al., 1985), Kolaskar and Tongaonkar Antigenicity (Kolaskar and Tongaonkar, 1990), Parker Hydrophilicity prediction (Parker et al., 1986) and Bepipred Linear Epitope prediction (Larsen et al., 2006)].

TABLE 2
Overview of field sera used for the validation of the PmpC assay.

| | Field sera | | | | |
|---|---|---|---|---|---|
| | | C. suis PCR | C. suis PCR | C. suis PCR | |
| Group | | vaginal swab | rectal swab | eye swab | |
| (No of | Clinical | (number of | (number of | (number of | Result |
| pigs) | signs | positive pigs) | positive pigs) | positive pigs) | culture |
| 6A (20) | No information | Positive (20) | Negative (20) | NA^{a} | NP^{b} |
| 6B (10) | No information | Negative (10) | Negative (10) | NA | NP |
| 7 (10) | Reproductive | Positive (3^{d}) | Positive (3^{d}) | NA | Positive (3^{d}) |
| | failure in sows^{c} | | | | |
| 8 (10) | Reproductive | NA | Positive (10) | Positive (10) | NP |
| | failure and | | | | |
| | conjunctivitis | | | | |
| | in boars^{e} | | | | |

^{a}NA: not applicable;
^{b}NP not performed;
^{c}sows showed decreased conception rate (went from 90% to <65%), white to yellow, non-smelling vulval discharge for about 1 week, low number of born piglets (2 to 5), non-uniform piglet weight (1 to 1.5 kg); irregular return to oestrus;
^{d}the same 3 animals;
^{e}boars showed inferior semen quality and conjunctivitis.

TABLE 3
Accession number of all PmpC coding sequences (CDS) used
for the selection of the C. suis-specific regions.

| Species | Strain | pmpC |
|---|---|---|
| C. trachomatis | A/HAR-13 | ctahar_446 |
| C. trachomatis | C/TW-3 | ctw3_02260 |
| C. trachomatis | L2/434/BU | ct434bu_703 |
| C. trachomatis | L1/115 | L1115_429 |
| C. trachomatis | D/13-96 | O176_02250 |
| C. trachomatis | E/150 | cte150_434 |
| C. trachomatis | F/6-94 | O172_02245 |
| C. trachomatis | G/9768 | ctg9768_433 |
| C. trachomatis | J/31-98 | O180_02245 |
| C. suis | MD56 | Q499_0437 |

In addition, the 9 Pmp regions within the selected Pmp were also used for serum antibody-based B cell epitope mapping. Hereto, 226 synthetic peptides (Pepscan Presto; Lelystad, The Netherlands) were synthetized. Peptides were eight amino acid residues in length, with an overlap of six residues. Peptides were pHPLC purified (purity up to 99%) and analyzed by MS-UPLC. All peptides contained an N-terminal acetyl group and were C-terminal attached to polyethylene pins via incorporation of an extra cysteine. Hundred μg peptide was coupled to each pin. The peptide coated pins were assembled on a 96-well polyethylene carrier (pin peptide ELISA format).

TABLE 4
Amino acid sequence and location of the nine C. suis-specific regions within
PmpC*. Linear epitope prediction results are underlined.

| | Position in | |
|---|---|---|
| | protein | |
| Amino acid sequence | sequence | SEQ ID NO |
| QEENEETDEDSSLDSRNT | 85-102 | 25 |
| AESAPEEPSSSTTTASQSTSSDQPQNNAIALRSFLYSLQTE | 126-166 | 26 |
| LTFSKNAVKVTFVDKSETQNPSGGSGTGDSSDSSEAEGSSGSSN | 283-369 | 27 |
| DSANNSSGGDSNGVSAAAQAAAFSRFLSASTSTDPQPGEAEN | | |
| GSQGTEQDSQEGSPGSTGSQESATNSASSQQSSIASARLTQLSL | 454-529 | 28 |
| TRSASAESGGSQSQENTSQQN VQASNDTQQGQ | | |
| APENNRQGDASENHQQGEQASSTENQNDNQSASENDQSSGG | 549-617 | 29 |
| GSSSPTSPQSPTTEVIKPVVGRGGAVYT | | |
| SGSEEASEASSGQEGGNEEGSASNEPQASPVRSSSEDRAQEAG | 965-1018 | 30 |
| SDSTPSSETN | | |
| AIPADNNSVTATVSDSGTPSTTPDP | 1286-1310 | 31 |
| PEGSGGSAASNTGSGSSGSGQGDNNAGSGSSSSTSTDNSN | 1630-1664 | 32 |
| SSSSSAPASSSTVPAA | 1781-1796 | 33 |

*PmpC of the C. suis MD56 strain contains 2140 amino acids.

Positive (groups 1, 2 and 4) and negative (group 3) control sera as well as sera of group 5 were used in the pin peptide ELISA to identify linear B cell epitopes in the 9 Pmp regions. First, pins were blocked for 3 hours at room temperature at 100 rpm using 250 μl per well of 0.01 M PBS (1.07 g/l Na₂HPO₄ (Sigma); 0.39 g/l NaH₂PO₄.2H₂O (VWR, Leuven, Belgium); 8.5 g/l NaCl (Acros Organics, Geel, Belgium); pH 7.2)+5% BSA (Sigma)+0.1% Tween 20 (Sigma). Afterwards, the pins were washed 3 times for 10 minutes at 100 rpm with 0.01 M PBS+0.1% Tween 20. After washing, pins were incubated overnight at 4° C. at 100 rpm with 150 μl serum per well. Sera were tested at a dilution of 1:100 and 1:500 in 0.01 M PBS+3% BSA+0.1% Tween 20. After the washing steps, pins were incubated for 1 hour at room temperature at 100 rpm with 150 μl per well of the rabbit anti-mouse-HRP conjugate, diluted at 1/1000 in 0.01 M PBS+3% BSA+0.1% Tween 20. Following the last washing steps, pins were incubated at room temperature at 100 rpm with 150 μl per well of the substrate H₂O₂-chromogen ABTS mixture (KPL, Gaithersburg, Md., USA) and the maximum absorbance at 405 nm could be determined. Results were expressed as the optical density (OD₄₀₅) after background subtraction for each individual pin-coated well. The mean background for all the pin-peptides, without adding serum was 0.052.

4. Evaluation of the Pmp Peptide Assay with Experimental Sera

The sensitivity and the C. suis-specificity of the PmpC ELISA were determined using experimental sera (Table 1). Sera were tested using the selected B cell epitope sequence attached to pins. The peptide representing the B cell epitope was eight amino acid residues in length. The peptide was pHPLC purified (purity up to 99%) and analyzed by MS-UPLC. The peptide contained an N-terminal acetyl group and was C-terminal attached to polyethylene pins via incorporation of an extra cysteine. A total of 100 μg of the peptide was coupled to each pin. The peptide coated pins were assembled on a 96-well polyethylene carrier (pin peptide ELISA format). Experimental sera were tested as described for B cell epitope mapping testing the field sera at a fixed dilution of 1:100.

The following formulas were used for calculation of specificity and sensitivity: sensitivity: =[TP/(TP+FN)]×100, where TP is the true positive result and FN is the false-negative result, and specificity=[TN/(TN+FP)]×100, where TN is the true negative result and FP is the false-positive result.

### 5. Validation of the Pmp Peptide Assay Using Field Sera

Field sera (group 6A, 6B, 7 and 8; Table 2) were used for validation of the PmpC antibody ELISA. Sera were tested as described for B cell epitope mapping testing the field sera at a fixed dilution of 1:100. Experimental sera of groups 2 and 3 served as positive and negative control sera, respectively. The cut-off value was the mean OD₄₀₅ of negative control sera±twice the standard deviation.

### 6. Statistics

Statistical analysis was conducted using IBM SPSS Statistics for Windows, Version 21.0 (IBM Corp., Armonk, N.Y.). Data are represented as mean value±standard deviation (SD). One-way ANOVA was used to compare the mean OD₄₀₅ of the positives between groups. Results were considered significantly different at P<0.05.

### Results

### 1. B Cell Epitope Mapping of PmpC

Linear epitope prediction revealed a total of nine peptides spread over 8 out of 9 C. suis-specific regions (Table 4). Chlamydia suis positive control sera (groups 1, 2 and 4) confirm the previously identified pin-peptide SQQSSIAS as the most sensitive B cell epitope, revealing the highest OD₄₀₅ values in ELISA.

Field sera of group 6B reacted negative in the pin-ELISA. However, unexpectedly, we have identified 13 further epitopes (SEQ ID 1 to 13) in said PmpC by testing field sera collected in the slaughterhouse from healthy pigs being C. suis positive in the vagina (group 6A) and by using field sera of clinically affected pigs being C. suis PCR and/or C. suis culture positive in either their reproductive tract or their conjunctiva (groups 7 and 8). As such, the presently identified epitopes are useful for the detecting C. suis infections and are of particular value for confirming C. suis as the etiological agent in reproductive failure and conjunctivitis.

### Discussion

Chlamydia suis infections, and especially infections with Tc^{R} C. suis strains are emerging in the pork industry and evidence for the transmission of C. suis to humans exists. The present study aimed at the identification of a C. suis-specific and immunogenic antigen for serodiagnosis and the validation of this antigen for use in a C. suis-specific assay. Serology cannot replace nucleic acid amplification tests, currently considered as the gold standard for Chlamydia diagnosis in both humans and animals. However, a C. suis-specific antibody ELISA for use in swine would be extremely useful as the test could be made applicable for direct use by veterinarians or animal care takers. Especially in swine, it is important to identify the Chlamydia species involved as tetracycline resistant C. suis strains have been found in pigs, while C. abortus, C. pecorum and C. psittaci, which can also infect pigs are, as far as we know, still tetracycline sensitive. Specific diagnosis allows proper treatment. Chlamydia may potentially cause persistent infections. During the immune response, intracellular tryptophan levels decrease as a consequence of IFNy-induced indol dioxigenase. Chlamydia are auxotrophic for tryptophan and respond to this stress situation with generation of morphological aberrant, non-replicative, persistent forms that presumably convert into replicative forms as environmental conditions improve. As for C. trachomatis (Meyer, 2016), which is phylogenetically highly related to C. suis, persistent C. suis infections most likely are associated with a positive antibody response, and thus negative serology may assist to rule out the involvement of chlamydia. In addition, as for C. trachomatis (Meyer, 2016), low-grade replication possibly also occurs during chronic C. suis infections and serology might be useful in the diagnostic work-up of such suspected chronic infections. However, serology is inappropriate for immediate diagnosis of acute C. suis infections as convalescent sera are needed to this purpose. In addition, automation for high throughput analysis would be possible by the current improvement in robotic and optical technology and the test could be used as a learning tool to study the pathogenesis and the kinetics of antibody production during C. suis infections in pigs. Additionally, diagnosis by ELISA is normally cheaper than molecular diagnosis, at least if the antigen (recombinant or synthetic) production costs can be kept to a minimum. Finally, from a biosafety point of view, the ELISA is safer to producers and end users as compared to tests based on cell-cultured antigen. As far as we know, there is no C. suis serological test for the specific diagnosis of this infection.

A number of surface-exposed chlamydial proteins/antigens contribute to the humoral response. Therefore, as for other ELISAs based on the use of a single antigen, the ELISA may give false negative results. However, regarding the sensitivity of the test, PmpC performed equally good as the well-known immunodominant C. suis MOMP.

The present invention identified B cell epitopes in PmpC using experimental sera and field sera.

The reference strain, C. suis S45 (ATCC VR1474) was isolated from feces of an asymptomatic pig in Austria in the late 1960s (Koelbl, 1969). This strain was tetracycline sensitive (Tc^{S}) as other chlamydial species. The enteric pathogenicity of the reference strain was demonstrated in gnotobiotic piglets. In addition, C. suis S45 was also capable of causing an ascending urogenital infection following an experimental vaginal inoculation in gilts (De Clercq et al., 2014). Nowadays, C. suis is often found in rectal swabs of sows and gilts (De Puysseleyr et al., 2017), suggesting that autoinoculation from the rectum to the genitals might occur in pigs. The latter has already been demonstrated for Chlamydia muridarum using orally infected mice (Yeruva et al., 2013). Chlamydia species can persist in the gastrointestinal (GI) tract for long periods of time in the absence of apparent inflammation and pathology (Rank and Yeruva, 2015). The PmpC antibody assay could assist in detecting those persistent asymptomatic intestinal C. suis infections (carriers) by use of the previously identified B cell epitope SQQSSIAS. In addition, SQQSSIAS could be used for a diagnostic confirmation of C. suis as etiology in diarrhea in pigs. On the other hand, it was demonstrated in the present invention that the newly identified epitopes are useful for a diagnostic confirmation of C. suis as the etiology of reproductive failure and conjunctivitis.

In conclusion, The PmpC based assay is an easily accessible assay to non-specialized laboratories. The test is also affordable to swine producers. The assay offers diagnostic opportunities as the test could assist in diminishing the spread of C. suis infections in the pork industry as it might be used in an attempt to realize a trade in C. suis sero-negative swine. Moreover, the assay could be used to prevent venereal transmission by serological monitoring of boars on the farms and in artificial insemination centers.

### REFERENCES

Andersen, A. A. and D. G. Rogers (1998). Resistance to tetracycline and sulfadiazine in swine C. trachomatis isolates. Ninth International Symposium on Human Chlamydial Infection, San Francisco, Calif.

Bocher, M., T. Boldicke, et al. (1997). “Synthesis of mono- and bifunctional peptide dextran conjugates for the immobilization of peptide antigens on ELISA plates: properties and application.” Journal of Immunological Methods 208(2): 191-202.
Borel, N., N. Regenscheit, et al. (2012). “Selection for tetracycline-resistant Chlamydia suis in treated pigs.” Vet Microbiol 156(1-2): 143-146.
Cevenini, R., M. Donati, et al. (1991). “Partial Characterization of an 89-Kda Highly Immunoreactive Protein from Chlamydia-Psittaci a/22 Causing Ovine Abortion.” Fems Microbiology Letters 81(1): 111-116.
Chahota, R., Ogawa, H., Ohya, K., Yamaguchi, T., Everett, K. D., Fukushi, H. (2017). Involvement of multiple Chlamydia suis genotypes in porcine conjunctivitis. Transboundary and Emerging Diseases, doi 10.1111/tbed. 12645
Chou, P. Y. and G. D. Fasman (1978). “Prediction of the secondary structure of proteins from their amino acid sequence.” Advances in enzymology and related areas of molecular biology 47: 45-148.
De Puysseleyr, K., L. De Puysseleyr, et al. (2014a). “Evaluation of the presence and zoonotic transmission of Chlamydia suis in a pig slaughterhouse.” Bmc Infectious Diseases 14: 560.
De Puysseleyr, K., L. De Puysseleyr, et al. (2014b). “Development and Validation of a Real-Time PCR for Chlamydia suis Diagnosis in Swine and Humans.” PloS one 9(5): e96704.
De Puysseleyr, L., De Puysseleyr, K., Braeckman, L., Morré, S. A., Cox, E., Vanrompay, D. (2017). Assessment of Chlamydia suis infection in pig farmers. Transboundary and Emerging Diseases. 64(3), 826-833
Dean, D., J. Rothschild, et al. (2013). “Zoonotic Chlamydiaceae Species Associated with Trachorna, Nepal.” Emerging Infectious Diseases 19(12): 1948-1955.
De Clercq, E., Devriendt, B., Yin, L., Chiers, K., Cox, E., Vanrompay, D. (2014). The immune response against Chlamydia suis genital tract infection partially protects against re-infection. Veterinary Research, 45:95
Di Francesco, A., M. Donati, et al. (2011). “Seroepidemiologic Survey for Chlamydia suis in Wild Boar (Sus scrofa) Populations in Italy.” J of Wildlife Dis 47(3): 709-712.
Donati, M., A. Di Francesco, et al. (2009). “In vitro detection of neutralising antibodies to Chlamydia suis in pig sera.” Veterinary Record 164(6): 173-174.
Eggemann, G., M. Wendt, et al. (2000). “Prevalence of Chlamydia infections in breeding sows and their importance in reproductive failure.” DTW. Deutsche tierarztliche Wochenschrift 107(1): 3-10.
Emini, E. A., J. V. Hughes, et al. (1985). “Induction of Hepatitis—a Virus-Neutralizing Antibody by a Virus-Specific Synthetic Peptide.” Journal of Virology 55(3): 836-839.
Forsbach-Birk, V., C. Foddis, et al. (2013). “Profiling Antibody Responses to Infections by Chlamydia abortus Enables Identification of Potential Virulence Factors and Candidates for Serodiagnosis.” PloS one 8(11).
Grimwood, J. and R. S. Stephens (1999). “Computational analysis of the polymorphic membrane protein superfamily of Chlamydia trachomatis and Chlamydia pneumoniae.” Microbial & comparative genomics 4(3): 187-201.

Hoelzle, L. E., K. Hoelzle, et al. (2004). “Recombinant major outer membrane protein (MOMP) of Chlamydophila abortus, Chlamydophila pecorum, and Chlamydia suis as antigens to distinguish chlamydial species-specific antibodies in animal sera.” Veterinary Microbiology 103(1-2): 85-90.
Ivanov, V. S., Z. K. Suvorova, et al. (1992). “Effective Method for Synthetic Peptide Immobilization That Increases the Sensitivity and Specificity of Elisa Procedures.” Journal of Immunological Methods 153(1-2): 229-233.
Joseph, S. J., Marti, H., Didelot, X., Read, T. D., Dean, D. (2016). Tetracycline selective pressure and homologous recombination shape the evolution of Chlamydia suis: a recently identified zoonotic pathogen. Genome Biology and Evolution, 8, 2613-2623
Karplus, P. A. and G. E. Schulz (1985). “Prediction of Chain Flexibility in Proteins—a Tool for the Selection of Peptide Antigens.” Naturwissenschaften 72(4): 212-213.
Koelbl, O. (1969). Untersuchungen Ober das Vorkommen von Miyagawanellen beim Schwein. Wiener Tierärztliche Monatsschrift, 56, 355-361.
Kolaskar, A. S. and P. C. Tongaonkar (1990). “A Semiempirical Method for Prediction of Antigenic Determinants on Protein Antigens.” Febs Letters 276(1-2): 172-174.
Larsen, J. E., O. Lund, et al. (2006). “Improved method for predicting linear B-cell epitopes.” Immunome research 2: 2.

Lis, P., A. Kumala, et al. (2014). “Novel locked nucleic acid (LNA)-based probe for the rapid identification of Chlamydia suis using real-time PCR.” Bmc Veterinary Research 10.
Longbottom, D. and L. J. Coulter (2003). “Animal chlamydioses and zoonotic implications.” Journal of Comparative Pathology 128(4): 217-244.
Longbottom, D., S. Fairley, et al. (2002). “Serological diagnosis of ovine enzootic abortion by enzyme-linked immunosorbent assay with a recombinant protein fragment of the polymorphic outer membrane protein POMP90 of Chlamydophila abortus.” Journal of Clinical Microbiology 40(11): 4235-4243.

Longbottom, D., E. Psarrou, et al. (2001). “Diagnosis of ovine enzootic abortion using an indirect ELISA (rOMP91B iELISA) based on a recombinant protein fragment of the polymorphic outer membrane protein POMP91B of Chlamydophila abortus.” Fems Microbiology Letters 195(2): 157-161.
Marques, P. X., P. Souda, et al. (2010). “Identification of immunologically relevant proteins of Chlamydophila abortus using sera from experimentally infected pregnant ewes.” Clinical and vaccine immunology: CVI 17(8): 1274-1281.
Meyer, T. (2016). Diagnostic procedures to detect Chlamydia trachomatis infections. Microorganisms, 4, 25.
Niessner, A., C. Kaun, et al. (2003). “Polymorphic membrane protein (PMP) 20 and PMP 21 of Chlamydia pneumoniae induce proinflammatory mediators in human endothelial cells in vitro by activation of the nuclear Factor-kappa B pathway.” Journal of Infectious Diseases 188(1): 108-113.
Novak, M., Z. Moldoveanu, et al. (1993). “Murine Model for Evaluation of Protective Immunity to Influenza-Virus.” Vaccine 11(1): 55-60.
Parker, J. M. R., D. Guo, et al. (1986). “New Hydrophilicity Scale Derived from High-Performance Liquid-Chromatography Peptide Retention Data—Correlation of Predicted Surface Residues with Antigenicity and X-Ray-Derived Accessible Sites.” Biochemistry 25(19): 5425-5432.
Rahman, K. S., Chowdhury, E. U., Poudel, A., Ruettger, A., Sachse, K., Kaltenboeck, B. (2015). Defining species-specific immunodominant B cell epitopes for molecular serology of Chlamydia species. Clinical Vaccine Immunology, 22(5), 539-52
Rank, R. G., & Yeruva, L. (2015) An alternative scenario to explain rectal positivity in Chlamydia-infected individuals. Clinical Infectious Diseases. 60(10), 1585-6.
Sachse, K., E. Vretou, et al. (2009). “Recent developments in the laboratory diagnosis of chlamydial infections.” Veterinary Microbiology 135(1-2): 2-21.
Schautteet, K., D. S. Beeckman, et al. (2010). “Possible pathogenic interplay between Chlamydia suis, Chlamydophila abortus and PCV-2 on a pig production farm.” The Veterinary record 166(11): 329-333.
Schautteet, K., E. De Clercq, et al. (2013). “Tetracycline-resistant Chlamydia suis in cases of reproductive failure on Belgian, Cypriote and Israeli pig production farms.” Journal of medical microbiology 62: 331-334.
Schautteet, K., E. Stuyven, et al. (2011). “Protection of pigs against Chlamydia trachomatis challenge by administration of a MOMP-based DNA vaccine in the vaginal mucosa.” Vaccine 29(7): 1399-1407.
Schautteet, K. and D. Vanrompay (2011). “Chlamydiaceae infections in pig.” Veterinary research 42(1): 29.
Tan, C., R. C. Hsia, et al. (2009). “Chlamydia trachomatis-Infected Patients Display Variable Antibody Profiles against the Nine-Member Polymorphic Membrane Protein Family.” Infection and Immunity 77(8): 3218-3226.
Vanrompay, D., Ducatelle, R., Haesebrouck, F. (1992). Diagnosis of avian chlamydiosis: specificity of the modified Giménez staining on smears and comparison of the sensitivity of isolation in eggs and three different cell cultures. Zentralblatt für Veterinärmedizin B, 39(2), 105-12.
Vanrompay, D., T. Geens, et al. (2004). “Immunoblotting, ELISA and culture evidence for Chlamydiaceae in sows on 258 Belgian farms.” Veterinary Microbiology 99(1): 59-66.
Verminnen, K., M. Van Loock, et al. (2006). “Evaluation of a recombinant enzyme-linked immunosorbent assay for detecting Chlamydophila psittaci antibodies in turkey sera.” Veterinary research 37(4): 623-632.
Wanninger, S., Donato, M., Di Francesco, A., Hässig, M., Hoffmann, K., Seth-Smith, H. M., Marti, H., Borel, N. (2016). Selective pressure promotes tetracycline resistance of Chlamydia suis in fattening pigs. PLosOne, 28, 11(11), e0166917
Wang, J., L. L. Chen, et al. (2009). “A chlamydial type III-secreted effector protein (Tarp) is predominantly recognized by antibodies from humans infected with Chlamydia trachomatis and induces protective immunity against upper genital tract pathologies in mice.” Vaccine 27(22): 2967-2980.
Wang, J., Y. Q. Zhang, et al. (2010). “Immunodominant Regions of a Chlamydia trachomatis Type III Secretion Effector Protein, Tarp.” Clinical and Vaccine Immunology 17(9): 1371-1376.
Wehrl, W., V. Brinkmann, et al. (2004). “From the inside out—processing of the Chlamydial autotransporter PmpD and its role in bacterial adhesion and activation of human host cells.” Molecular Microbiology 51(2): 319-334.
Yeruva, L., Spencer, N., Bowlin, A. K., Wang, Y., Rank, R. G. (2013). Chlamydial infection of the gastrointestinal tract: a reservoir for persistent infection. Pathogens and Disease, 68(3), 88-95.

1.-10. (canceled)

11. An in vitro method for detecting a presence of Chlamydia suis antibodies in a pig, the method comprising:
providing a biological sample from the pig, and
analyzing the biological sample for the presence of antibodies against one or more peptides selected from the group consisting of SEQ ID NO: 1-13, wherein the method does not comprise use of full-length polymorphic membrane protein C (PmpC) of Chlamydia suis.

12. The method according to claim 11, wherein the presence of the antibodies against one or more peptides is indicative for a past, early or late Chlamydia suis infection in a vagina or reproductive tract of the pig.

13. The method according to claim 11, wherein the biological sample from the pig is a body fluid.

14. The method according to claim 11, wherein the biological sample from the pig is a mucosal secretion or blood.

15. The method according to claim 11, wherein the biological sample from the pig is serum.

16. The method according to claim 11, wherein the antibodies detected are of an IgA, IgM and/or IgG type.

17. The method according to claim 11, wherein the presence of the antibodies is detected by chromatography, mass spectrometry, enzymatic assays, electrophoresis and/or antibody-based assays.

18. The method according to claim 11, wherein the presence of the antibodies is detected via an immune assay selected from the group consisting of: Enzyme Immuno Assay (EIA), Radio Immuno Assay (RIA), Immunoblotting, Enzyme Linked ImmunoSorbent Assay (ELISA), ChemiLuminescent Immuno Assay (CLIA), Cloned Enzyme Donor Immunoassay (CEDIA), Chemiluminescent Microparticle Immunoassay (CMIA), Microparticle Enzyme Immunoassay (META), Fluorescence Polarization Immunoassay (FPIA), Gold-Labeled, Optically read, Rapid Immunoassay (GLORIA), microarray analysis, fully-automated or robotic immunoassays, and latex agglutination assays.

19. A kit for diagnosis of a Chlamydia suis infection in pigs, the kit comprising an isolated peptide of 8 to 30 amino acids long comprising one or more peptides selected from the group consisting of SEQ ID NO: 1-13, wherein the peptides are immobilized on a solid support.

20. The kit according to claim 19, wherein at least two of the peptides are coupled, and are part of the amino acid sequence selected from the group consisting of SEQ ID NO: 14-24, or a sequence at least 75% identical to SEQ ID NO: 1-24.

21. The kit according to claim 19, wherein the kit further comprises a detection agent.

22. A method for diagnosing a Chlamydia suis infection in pigs having reproductive failure or conjunctivitis, the method comprising:
providing a biological sample from the pig, and
analyzing the biological sample for a presence of antibodies against one or more peptides selected from the group consisting of SEQ ID NO: 1-13, wherein the method does not comprise use of the full-length polymorphic membrane protein C (PmpC) of Chlamydia suis, and wherein the presence of the antibodies to one or more of the peptides is indicative of a Chlamydia suis infection in the pig.

23. A method for preparing a peptide of 8 to 30 amino acids long comprising one or more of the peptides selected from the group consisting of SEQ ID NO: 1-13, wherein the peptides are produced by synthetic chemistry, polypeptide hydrolysis or recombinant technology.
